(19) United States
(12) Patent Application Publication
(10) Pub. No.: US 2021/0020295 A1
(43) Pub. Date: Jan. 21, 2021

(54) PHYSICAL FUNCTION INDEPENDENCE SUPPORT DEVICE OF PHYSICAL FUNCTION AND METHOD THEREFOR

(71) Applicant: HITACHI, LTD., Tokyo (JP)

(72) Inventors: Yuan LI, Tokyo (JP); Shinya YUDA, Tokyo (JP); Hideaki SUZUKI, Tokyo (JP)

(73) Assignee: HITACHI, LTD., Tokyo (JP)

(21) Appl. No.: 16/981,608

(22) PCT Filed: Mar. 30, 2018
(86) PCT No.: PCT/JP2018/013861

§ 371 Date: Sep. 16, 2020

(51) Int. Cl. G16H 20/30; G16H 50/30; G16H 50/70; G16H 50/20; G16H 50/50; G09B 19/00; A61B 5/11; A61B 5/00

(52) U.S. Cl. CPC G16H 20/30; G16H 50/30; G16H 50/70; G16H 50/20; A61B 5/486; G09B 19/00; A61B 5/1116; A61B 5/1118; A61B 5/743; G16H 50/50

(57) ABSTRACT

Provided are: an acquisition unit that acquires physical state information indicating physical states of the people from one or more sensors that detect at least the people; a physical function analysis unit that analyzes a change in physical functions of the people based on a time-series change of the physical state information acquired by the acquisition unit; and a physical function improvement proposing unit that generates and outputs physical function improvement proposal information indicating an improvement proposal of the physical function with respect to the change in the physical functions of the people based on an analysis result of the physical function analysis unit.

### TECHNICAL FIELD

The present invention relates to a physical function independence support device that analyzes a physical function and supports maintenance or improvement of the physical function, and a method therefor.

### BACKGROUND ART

In nursing care business, various services and facilities are provided to elderly who need nursing care. For example, the market for home nursing care, medical services, nursing homes for the elderly, insurance facilities, recuperation facilities, group homes, day care, and the like has been very mature. There is a clear demand for caregivers to perform health checkups, management, daily life support, and the like for the elderly. However, the support for the elderly by caregivers requires a large amount of resources.

In recent years, the market for nursing care of the elderly has been expanded with an increase in the elderly population over the age of 65. In the nursing care business, services have expanded not only for elderly people who need nursing care, but also for elderly people who may need nursing care in the future and healthy elderly people. Accordingly techniques that measure states of the elderly have been developed. If measurement items are roughly classified, there are a technique that measures body dimensions such as body shape information, height, and weight, and a technique that measures physical function information.

As the conventional technique that measures physical function information, there are a technique that uses a wearable sensor to measure a heart rate, a blood pressure, electroencephalogram, and the like, and a technique that uses a non-contact sensor to digitize and measure human motion and posture.

As the technique that digitizes and measures the human motion using the non-contact sensor, there is a motion capture technique that digitizes a human motion by attaching a marker to a joint or the like and processing information of the detected marker. Further, there is also a technique that extracts position information and skeleton information of a person by image processing and detects a behavior of the person such as walking and standing still. Further, a deep learning technique has developed, and it is possible to extract a plurality of skeletons of a person from an image captured by a monocular camera when a dedicated active sensor is used instead of a marker, and a posture can be digitally measured.

As a device using these techniques, there is a device that processes a walking motion described in PTL 1. This device extracts a skeleton of a person, a position of a landing point, and movement track information to provide information for evaluating a walking state. The information for evaluating the walking state can be used for rehabilitation. For example, in the rehabilitation, coordinate information of each joint of a pedestrian can be extracted, and information such as a walking speed and a step length can be displayed without blurring.

Further, there is a technique that digitizes and measures not only a motions and a posture of a person, such as walking and standing still, but also a daily activity. For example, in a human motion monitoring method and motion determination method described in PTL 2, information such as a motion and a position of a person can be extracted using a plurality of sensors, and it is possible to grasp daily activities of the elderly for care. Further, the information is visualized so that an abnormal behavior such as fall and slip is predicted and presented to a caregiver in accordance with position information and facility information. For example, when it is determined that an elderly person has moved to a place with a step, it is possible to display an assistance for moving on the step to the caregiver and prevent a risk.

### CITATION LIST

### Patent Literature

PTL 1: JP 2015-042241 A
PTL 2: JP 2016-66308 A

### SUMMARY OF INVENTION

### Technical Problem

Conventionally, as an independence support system configured to improve the healthy life expectancy of the elderly, no proposal has been made regarding a system that considers “(1) to grasp and analyze “what can be done” by the elderly with their physical functions, and (2) to maintain or improve the ability of physical functions so as to smoothly perform “what can be done””

PTL 1 describes a method for facilitating evaluation of a motion and a posture such as walking of a person, but does not describe a method for maintaining or improving walking ability. Further, the daily activity of the person is measured in PTL 2, but there is no proposal for maintenance or improvement of the person's health in order to improve the person's health rather than the daily activity.

That is, in the conventional measurement methods, information of a cared person is properly digitized such that a caregiver can easily take care of the cared person. This is because it is understood that the caregiver can provide support to the cared person if having the information. Here, it is an absolute requirement that the caregiver has expertise. It is difficult for the elderly to grasp their own physical states since they do not have expertise. It is more difficult for one to improve his/her own health. It is not possible to become independent by receiving training according to expert's opinions as in the related art.

An object of the present invention is to provide information for support of independent health management.

### Solution to Problem

In order to achieve the above object, the present invention provides a physical function independence support device which transmits and receives information to and from one or more sensors that detect at least people, the physical function independence support device including: an acquisition unit that acquires physical state information indicating physical states of the people from the sensor; a physical function analysis unit that analyzes a change in physical functions of the people based on a time-series change of the physical state information acquired by the acquisition unit; and a physical function improvement proposing unit that generates and outputs physical function improvement proposal information indicating an improvement proposal of the physical function with respect to the change in the physical functions of the people based on an analysis result of the physical function analysis unit.

### Advantageous Effects of Invention

According to the present invention, it is possible to provide the information for support of the independent health management.

### BRIEF DESCRIPTION OF DRAWINGS

FIG. 1 is a configuration diagram illustrating a configuration example of a physical function independence support system according to a first embodiment of the present invention.

FIG. 2 is a configuration diagram illustrating a configuration of software resources of a server according to the first embodiment of the present invention.

FIG. 3 is a configuration diagram illustrating a configuration example of a database S 18 according to the first embodiment of the present invention.

FIG. 4 is a configuration diagram illustrating a display example of an analysis result of a physical state analysis unit 12 according to the first embodiment of the present invention.

FIG. 5 is a configuration diagram illustrating a specific configuration of a physical function analysis unit 14 according to the first embodiment of the present invention.

FIG. 6 is a configuration diagram illustrating a display example of an analysis result of the physical function analysis unit 14 according to the first embodiment of the present invention.

FIG. 7 is a configuration diagram illustrating an example of a configuration of physical function improvement proposal information according to the first embodiment of the present invention.

FIG. 8 is an explanatory diagram illustrating an example of creating a standard index according to the first embodiment of the present invention.

FIG. 9 is a configuration diagram illustrating a display example of information proposed by a physical function improvement proposing unit 16 according to the first embodiment of the present invention.

FIG. 10 is a flowchart illustrating a processing flow when performing health maintenance support for a healthy elderly person according to the first embodiment of the present invention.

FIG. 11 is a flowchart illustrating a processing flow when performing modulation management for an elderly person according to the first embodiment of the present invention.

FIG. 12 is a flowchart illustrating a processing flow when diagnosing and treating an elderly person according to the first embodiment of the present invention.

FIG. 13 is a configuration diagram illustrating a display example when performing physical function support for a group of elderly according to a second embodiment of the present invention.

FIG. 14 is a flowchart illustrating a processing flow when a plurality of elderly are divided into a plurality of groups and physical functions of the elderly people belonging to each of the groups are analyzed according to the second embodiment of the present invention.

FIG. 15 is a flowchart illustrating a processing flow when performing motor function support for exercise training of a child according to a third embodiment of the present invention.

FIG. 16 is a flowchart illustrating a processing flow when performing physical function support for work of a worker according to a fourth embodiment of the present invention.

### DESCRIPTION OF EMBODIMENTS

Hereinafter, embodiments of the present invention will be described in detail with reference to the drawings. In the following description of the embodiments of physical function independence support system and device of the present invention, a physical function independence support system for an elderly person will be described as an example.

### First Embodiment

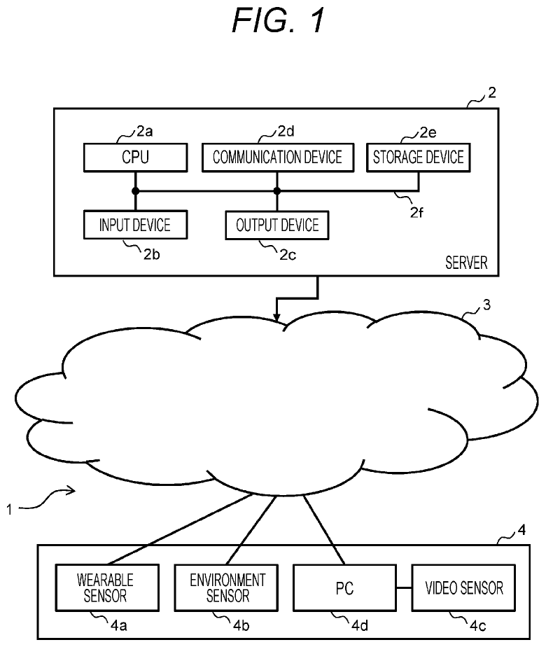

FIG. 1 illustrates a configuration example of a physical function independence support system according to a first embodiment of the present invention. In FIG. 1, the physical function independence support system 1 includes a server 2, a network 3, and one or more user terminals 4, and the server 2 is connected to the user terminal 4 via the network 3.

The server 2 is, for example, a computer device including a central processing unit (CPU) 2a, an input device 2b, an output device 2c, a communication device 2d, a storage device 2e, and a bus 2f, and is configured as a physical function independence support device. The CPU 2a, the input device 2b, the output device 2c, the communication device 2d, and the storage device 2e are connected to each other via the bus 2f. The CPU 2a is configured as a controller (central processing unit) that controls the operation of the entire server in an integrated manner. The input device 2b is configured using a keyboard or a mouse, and the output device 2c is configured using a display or a printer. Further, these may be configured using a smart device such as a tablet having the same function. The communication device 2d includes, for example, a network interface card (NIC) for connection to a wireless local area network (LAN) or a wired LAN. Furthermore, the storage device 2e includes a storage medium such as a random access memory (RAM), a read only memory (ROM), and a hard disk drive (HDD).

The user terminal 4 includes: a plurality of sensors that detect at least a person (an elderly person, a child, a worker, and the like), for example, a wearable sensor 4a, an environment sensor 4b, and a video sensor 4c, and further includes a personal computer (PC) 4d. The wearable sensor 4a and the environment sensor 4b are connected to the server 2 via the network 3, and the video sensor 4c is connected to the server 2 via the personal computer (PC) 4d. The personal computer (PC) 4d is configured using, for example, a computer device including a CPU, a memory, an input/output interface, a display (all not illustrated), and the like. It should be noted that the wearable sensor 4a and the environment sensor 4b can be also connected to the server 2 via the personal computer (PC) 4d.

The wearable sensor 4a is a sensor that is worn on a body of a person who is a target of physical function independence support, for example, an elderly person, and measures physical state information regarding a physical state of the elderly person. Examples of the wearable sensor 4a include a heartbeat sensor, a blood pressure sensor, an electroencephalogram sensor, and the like. These sensors can receive a physiological signal from the body of the elderly person. The multiple wearable sensors 4a are installed in an acceleration sensor, a pulse wave sensor, a body temperature sensor, and the like.

The environment sensor 4b is a sensor that depends on the body of the elderly person and collects information on the environment. Examples of the environment sensor 4b include a global positioning system (GPS) sensor that grasps position information, a voice sensor that senses a voice of the elderly person, a sensor that detects information on weather, such as a temperature sensor that detects temperature, an atmospheric pressure sensor that detects atmospheric pressure, and a humidity sensor that detects humidity, and the like.

The video sensor 4c is a sensor that can acquire an image (information) of the elderly person such as a monocular camera, a stereo camera, a time of flight (ToF) camera, and an active sensor. The monocular camera includes an image sensor and a lens. The image sensor is a mechanism including an imaging element such as a complementary metal oxide semiconductor (CMOS) and a charge coupled device (CCD). The lens is a zoomable lens or a fixed lens. The zoomable lens can image a far-side region and can also image a near-side region by zooming. The fixed lens can image a region within a certain range. The captured image is saved in a format such as BMP and JPGE. The image contains RGB information as color information. The stereo camera is a camera that can acquire a depth Depth by simultaneously performing capturing from a plurality of viewpoints. Further, the ToF camera is a sensor that can emit light, measure the time until the emitted light is reflected by an object and then received, and also measure the depth Depth together with the speed of the light. The monocular camera has no RGB information, which is different from the stereo camera. The active sensor is an RGBD sensor that can acquire the depth Depth in addition to an image RGB. These video sensors capture an appearance of the elderly person and generate captured data as image data.

It is possible to use one of the above-described sensors or a plurality of sensors in combination. It should be noted that these sensors may be either directly connected to the network 4 (the wearable sensor 4a and the environment sensor 4b in FIG. 1) or connected to the PC (the video sensor 4c in FIG. 1).

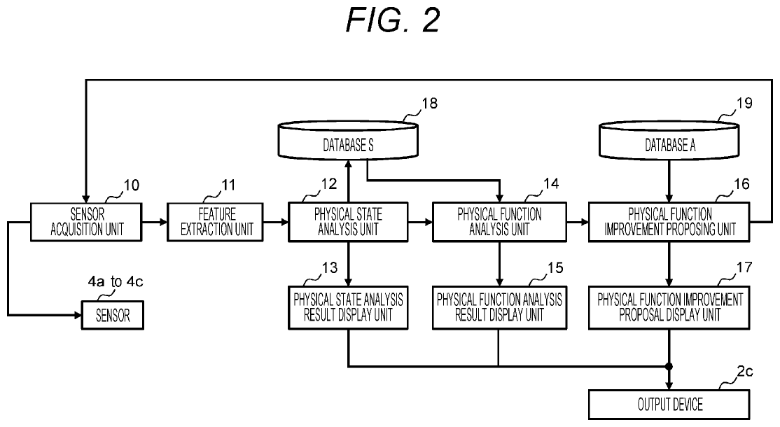

FIG. 2 is a block diagram illustrating a configuration of software resources of the server. In FIG. 2, the server 2 includes, as the software resources, a sensor acquisition unit 10, a feature extraction unit 11, a physical state analysis unit 12, a physical state analysis result display unit 13, a physical function analysis unit 14, a physical function analysis result display unit 15, a physical function improvement proposing unit 16, a physical function improvement proposal display unit 17, a database S 18, and a database A 19. At this time, the CPU 2a executes various processing programs stored in the storage device 2e, for example, a sensor acquisition program, a feature extraction program, a physical state analysis program, a physical state analysis result display program, a physical function analysis program, a physical function analysis result display program, a physical function improvement proposing program, and a physical function improvement proposal display program, thereby implementing functions of the sensor acquisition unit 10, the feature extraction unit 11, the physical state analysis unit 12, the physical state analysis result display unit 13, the physical function analysis unit 14, the physical function analysis result display unit 15, the physical function improvement proposing unit 16, and the physical function improvement proposal display unit 17.

It should be noted that each piece of information (data) detected by the wearable sensor 4a, the environment sensor 4b, and the video sensor 4c is acquired by the sensor acquisition unit 10, and image information generated by each of the physical state analysis result display unit 13, the physical function analysis result display unit 15, and the physical function improvement proposal display unit 17 is displayed on a screen of the output device 2c.

The sensor acquisition unit 10 acquires the information detected by the respective sensors, for example, physical state information indicating a physical state of a person, from the wearable sensor 4a, the environment sensor 4b, and the video sensor 4c, and outputs the acquired information to the feature extraction unit 11.

The feature extraction unit 11 extracts feature information, which is a feature, from the information (physical state information) input from the sensor acquisition unit 10, and outputs the extracted feature information to the physical state analysis unit 12. Here, the feature information extracted by the feature extraction unit 11 is defined as feature data. High cost is required in terms of a system to process the entire information detected by the respective sensors. Therefore, the feature extraction unit 11 extracts the feature data from the information (physical state information) input from the sensor acquisition unit 10 in order to reduce the cost. For example, when heart rate information is input as the physical state information from the wearable sensor 4a, data, which is higher and lower than standard data, is extracted as the feature data. When the physical state information is input from the video sensor 4c, the feature data can be extracted using RGB information of an image and information of depth Depth. Recently, deep learning has developed, and human detection, human recognition, behavior recognition, and behavior understanding on images can be more easily performed with high accuracy. For example, human skeleton information, foot position, position information, behavior information, and the like are all considered as the feature data. When the physical state information is input from the environment sensor 4b, abnormal weather information and the like can be extracted as the feature data. When the physical state information from a plurality of sensors is used, the feature data can be obtained from each piece of the physical state information.

The physical state analysis unit 12 uses the feature data extracted by the feature extraction unit 11 to compare measurement data obtained by measuring an elderly person at the current state, for example, with the feature data, and analyze the physical state of the elderly person. The physical state can be seen as a health index of the elderly person such as a daily activity, a motion, a posture, a physical fatigue level, and a physical burden level. For example, it is possible to analyze the posture of the elderly person using a foot position, a head position, and the like, which are the feature data extracted by the feature extraction unit 11. The physical state analysis unit 12 stores information indicating a result of the analysis using the feature data, for example, posture information in the database S (status) 18.

The database S (first database) 18 stores physical state analysis information indicating the analysis result of the physical state analysis unit 12. The information (physical state analysis information) for each elderly person can be stored in the database S 18 in association with an identification (ID) that identifies each elderly person. Using this ID, it is also possible to manage personal information related to health, such as height, weight, and history of the elderly person. Further, the database S 18 can also store information for each elderly person along the time axis.

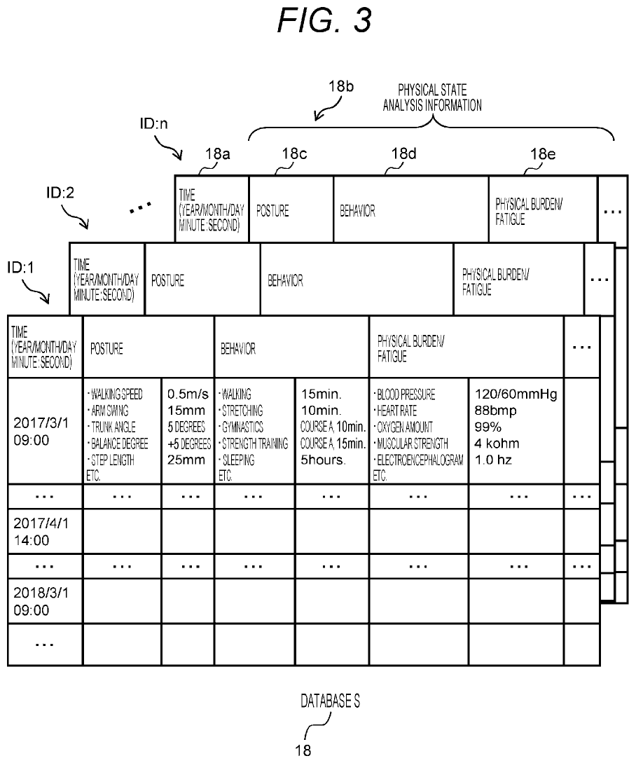

FIG. 3 illustrates an example of a configuration of the database S 18. The database S 18 is information stored in the storage device 2e and includes time 18a and physical state analysis information 18b, and the physical state analysis information 18b includes a posture 18c, a behavior 18d, and a physical burden/fatigue 18e, and is managed by IDs: 1 to n assigned to the respective elderly people. The physical state analysis information 18b obtained by analyzing the physical state of each elderly person is recorded in the database S 18.

In the time 18a, “year/month/day/minute: second” is recorded as information on the time when the physical state analysis unit 12 has analyzed the physical state of each elderly person. In the posture 18c, “walking speed”, “arm swing”, “trunk angle”, “balance degree”, “step length”, and the like are recorded as information on the posture of each elderly person. In the behavior 18d, “walking”, “stretching”, “gymnastics”, “strength training”, “sleeping”, and the like are recorded as information on the behavior of each elderly person. In the physical burden/fatigue 18e, “blood pressure”, “heart rate”, “oxygen amount”, “muscular strength”, “electroencephalogram”, and the like are recorded as information on the physical burden/fatigue of each elderly person.

The information (“walking speed”, “arm swing”, “trunk angle”, “balance degree”, and “step length”) that belongs to the posture 18c can be obtained by analyzing the feature data such as the foot position (foot position) and the head position extracted by the feature extraction unit 11. Further, the information (“walking”, “stretching”, “gymnastics”, “strength training”, and “sleeping”) that belongs to the behavior 18d can be obtained by analyzing the feature data such as the position information and the motion information extracted by the feature extraction unit 11, the feature data indicating a current behavior state. Examples of the feature data indicating the current behavior state include a walking time, stretching, gymnastics, training course and time, sleeping, sitting, falling, and the like. Further, the information (“blood pressure”, “heart rate”, “oxygen amount”, “muscular strength”, and “electroencephalogram”) that belongs to the physical burden/fatigue 18e can be obtained by analyzing the feature data obtained by measuring current blood pressure, heart rate, oxygen content, muscle strength, and electroencephalogram. The database S 18 can also store and manage the physical state information extracted from a plurality of sensors.

The physical state analysis unit result display unit 13 generates image information to visualize the analysis result of the physical state analysis unit 12, and displays the generated image information on the screen of the output device 2c. At this time, the physical state analysis unit result display unit 13 can display the analysis result in real time on the screen of the output device 2c, or search the designated time and display the analysis result at that time on the screen of the output device 2c.

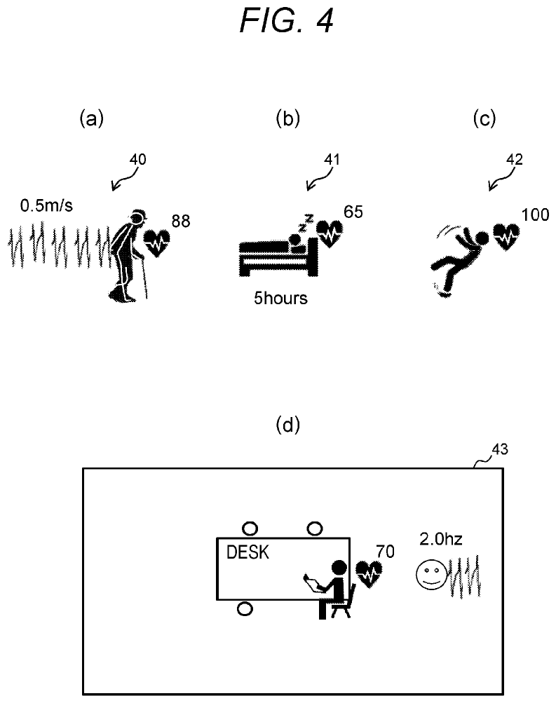

FIG. 4 illustrates a display example of the analysis result of the physical state analysis unit 12. A display screen 40 of FIG. 4(a) is displayed by combining analyzed walking speed and heart rate of an elderly person. To display skeletal information and video information together facilitates understanding of the elderly person. A display screen 41 of FIG. 4(b) displays sleeping behavior and heart rate of the elderly person. At this time, it is also possible to display a sleeping period of the elderly person. A display screen 42 of FIG. 4(c) displays falling behavior and heart rate when the elderly person falls. Further, it is possible to visualize the physical state analysis result in accordance with map information. For example, a display screen 43 of FIG. 4(d) displays a heart rate and electroencephalogram of the elderly person reading at a desk.

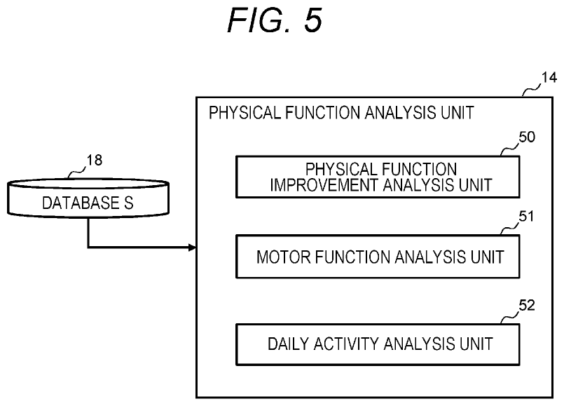

The physical function analysis unit 14 analyzes a change in the physical function of the person based on a time-series change of the physical state information acquired by the sensor acquisition unit 10. At this time, the physical function analysis unit 14 can hold the physical state information acquired by the sensor acquisition unit 10 in a time-series manner, and analyze the change in the physical function of the person based on the stored physical state information. Further, when using the feature information output from the feature extraction unit 11 that extracts the feature information, which is a feature, from the physical state information acquired by the sensor acquisition unit 10, the physical function analysis unit 14 analyzes the change in the physical function of the person based on a time-series change of the feature information extracted by the feature extraction unit 11. In this case, the speed of information processing can be increased as compared with a case where the feature information is not used. Further, when using the database S 18 that stores the physical state information acquired by the sensor acquisition unit 10 in a time-series manner, the physical function analysis unit 14 analyzes the change in the physical function of the person based on the feature information extracted by the feature extraction unit 11 and the physical state information stored in the database S 18. In this case, the speed of information processing can be increased as compared with a case where the feature information is not used. Further, the physical function analysis unit 14 analyzes the physical function of the elderly person using the analysis result of the physical state analysis unit 12 and the information of the physical state stored in a time-series manner (the physical state analysis information stored in the database S 18), and outputs an analysis result to the physical function analysis result display unit 15 and the physical function improvement proposing unit 16. FIG. 5 illustrates a specific configuration of the physical function analysis unit 14. The physical function analysis unit 14 includes a physical function improvement analysis unit 50, a motor function analysis unit 51, and a daily activity analysis unit 52.

The physical function improvement analysis unit 50 analyzes whether a physical state of an elderly person is improved based on the analysis result of the physical state analysis unit 12 and the data (physical state analysis information) recorded in the database S 18. The physical state is information recorded in the database S 18 and represents a health condition of an elderly person. The motor function analysis unit 51 comprehensively analyzes a type of exercise, burned calories, and exercise time based on the analysis result of the physical state analysis unit 12 and the behavior data recorded in the database S 18 (information belonging to the behavior 18d), and evaluates the appropriateness of the exercise based on analysis results. Further, an average heart rate, an average muscle strength, and an average blood pressure during the exercise of the elderly person are analyzed, so that the exercise ability of the elderly person can be evaluated. The daily activity unit 52 analyzes a daily activity of the elderly person based on the analysis result of the physical state analysis unit 12 and the data (physical state analysis information) recorded in the database S 18, and evaluates whether the daily life is changing suddenly based on an analysis result.

The physical function analysis unit 14 can change the analysis content by the function of evaluating the physical state of the elderly person. For example, the evaluation time series can be set in units of one week, one month, and one year to perform analysis. Further, when a rehabilitation effect is evaluated, it is necessary to illustrate the effect of rehabilitation basically for three months, and it is possible to compare physical states before and after rehabilitation to analyze whether the physical function of the elderly person has been improved.

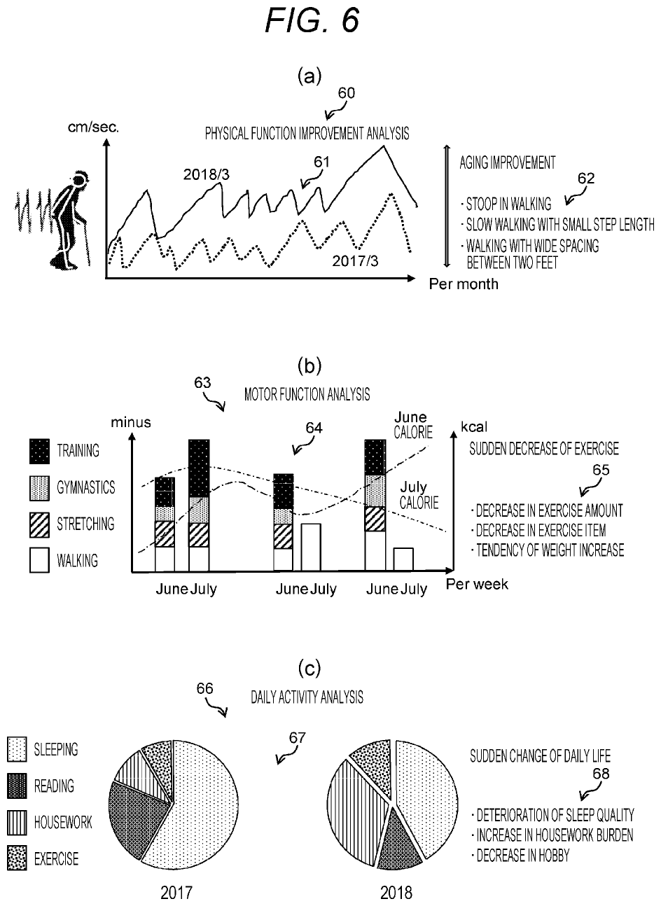

The physical function analysis result display unit 15 generates image information to visualize the analysis result of the physical function analysis unit 14, and displays the generated image information on the screen of the output device 2c. FIG. 6 illustrates a display example of the analysis result of the physical function analysis unit 14. A display screen 60 of FIG. 6(a) displays information on physical function improvement analysis 61 and an aging improvement 62. The physical function improvement analysis 61 displays information indicating a change in walking speed of an elderly person. Since display is performed based on the data obtained by storing the change in walking speed, the change in walking speed of the elderly person can be evaluated. The case of FIG. 6(a) illustrates that the walking speed in March 2018 has been significantly improved as compared with the walking speed in March 2017. A walking style of the elderly person has been evaluated based on the result evaluated here, and information of “stoop in walking”, “slow walking with small step length”, and “walking with wide spacing between two feet” is displayed as improved information in the aging improvement 62.

Further, a display screen 63 of FIG. 6(b) displays information on motor function analysis 64 and a sudden decrease of exercise 65. The motor function analysis 64 displays information on time and calories for the exercise items (“training”, “gymnastics”, “stretching”, and “walking”) of the elderly person. It was found that the monthly exercise decreased sharply by evaluating the exercise items and time every week. Such a change is displayed as information on the sudden decrease of exercise 65, for example, “decrease in exercise amount”, “decrease in exercise item”, and “tendency of weight increase”.

Further, a display screen 66 of FIG. 6(c) displays information on daily activity analysis 67 and a sudden change of daily life 68. In the daily activity analysis 67, information in 2017 and 2018 is displayed as information on the proportion of a daily activity (“sleeping”, “reading”, “housework”, and “exercise”) of the elderly person. The daily activities of one year are compared, and, if the daily life of the elderly person changes suddenly, a content of the change is displayed in the sudden change of daily life 68. For example, “deterioration of sleep quality”, “increase in housework burden”, and “decrease in hobby” are displayed in the sudden change of daily life 68. With these information, it is possible to analyze that mental and physical burdens will be imposed to the elderly person due to the sudden change of daily life.

When these analysis results are displayed, the adjustment in a time-series manner is possible. For example, when changes for one week, one month, and one year are displayed, tendency in the corresponding period can be analyzed. Further, it is also possible to analyze the influence on the physical function based on the change or tendency.

The physical function improvement proposing unit 16 generates and outputs physical function improvement proposal information indicating an improvement proposal of the physical function for the change in the physical function of the person based on the analysis result of the physical function analysis unit 14. For example, the physical function improvement proposing unit 16 compares the analysis result of the physical function analysis unit 14 with the information (standard index information indicating a standard index of physical state analysis information) recorded in the database A (second database) 19, and generates the physical function improvement proposal information based on a comparison result. Specifically, the physical function improvement proposing unit 16 generates information for maintenance or improvement of the physical function of the elderly person (physical function improvement proposal information) according to the information recorded in the database A 19 and the content analyzed by the physical function analysis unit 14, proposes the generated information automatically in terms of the system, and outputs the proposed content (physical function improvement proposal information) to the sensor acquisition unit 10 and the physical function improvement proposal display unit 17.

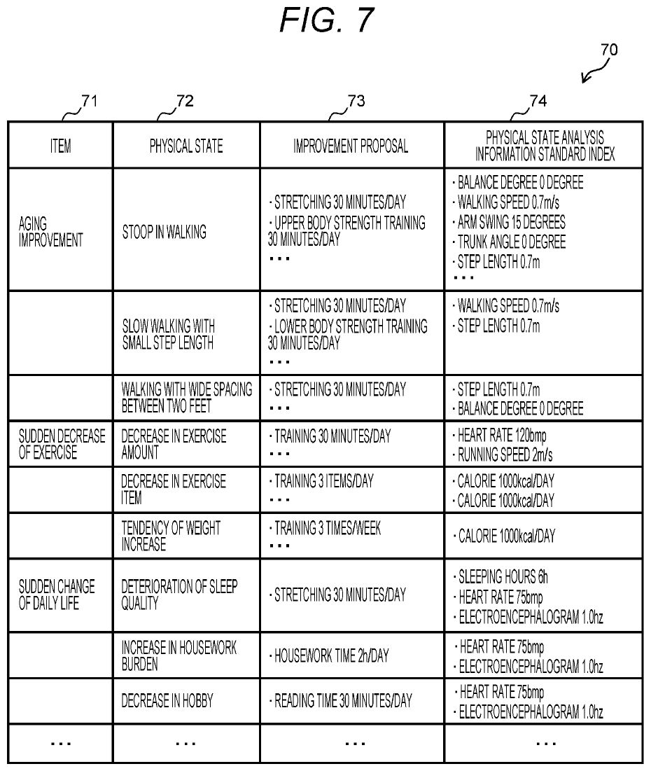

FIG. 7 illustrates an example of a configuration of the physical function improvement proposal information. Physical function improvement proposal information 70 includes an item 71, a physical state 72, an improvement proposal 73, and a physical state analysis information standard index 74. In the item 71, information on “aging improvement”, “sudden decrease of exercise”, and “sudden change of daily life” is recorded. In the physical state 72, as information associated with the item 71, for example, “stoop in walking”, “slow walking with small step length”, and “walking with wide spacing between two feet” are recorded in association with “aging improvement”. In the improvement proposal 73, as information associated with the item 71 and the physical state 72, for example, “stretching 30 minutes/day”, and “upper body strength training 30 minutes/day” are recorded in association with “aging improvement” and “stoop in walking”. In the physical state analysis information standard index 74, as information associated with the item 71 and the physical state 72, for example, “balance degree 0 degree”, “walking speed 0.7 m/s”, “arm swing 15 degree”, “trunk angle 0 degree”, and “step length 0.7 m” are recorded in association with “aging improvement” and “stoop in walking”.

A result analyzed by the physical function analysis unit 14 out of the physical function improvement proposal information 70 is reflected in the item 71 and the physical state 72. Then, the improvement proposal 73 can be pointed out according to the physical state 72. Further, information that belongs to the physical state analysis information standard index 74 is also presented for the information that belongs to the physical state 72. At this time, as the physical state analysis information standard index 74, standard data can be learned and a modeled index value can be used. This index value is an index that is to be desirably improved. The information that belongs to the improvement proposal 73 is information for maintenance or improvement of health with respect to this standard index. It should be noted that the physical function improvement proposing unit 16 generates information to support health maintenance of a person as the information belonging to the physical function improvement proposal information when information on the health maintenance of the person is included in the analysis result of the physical function analysis unit 14 and standard index information indicating a standard index for the health maintenance of the person is stored in the database A 19.

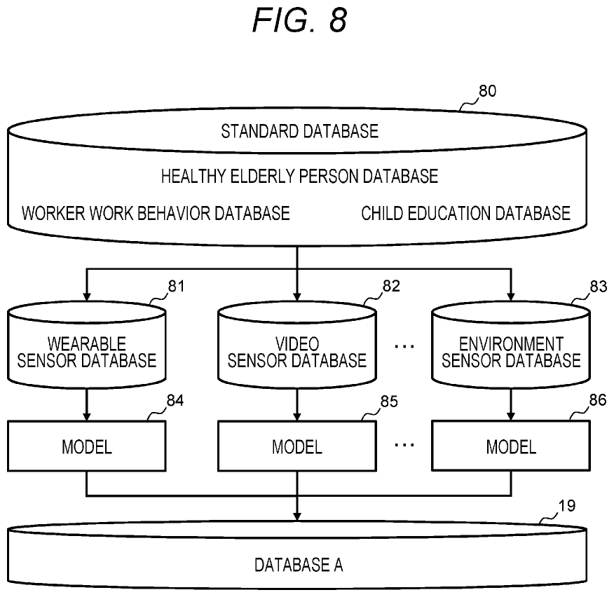

FIG. 8 illustrates an example of creating the standard index. A standard database 80 is a database managed by the physical function improvement proposing unit 16, and is configured as a database that stores data related to a worker work behavior database, a healthy elderly people database, and a child education database. Among pieces of data stored in the standard database 80, the data detected by the wearable sensor 4a is stored in a wearable sensor database 81, the data detected by the video sensor 4c is stored in an video sensor database 82, and the data detected by the environment sensor 4b is stored in an environment sensor database 83. The data extracted from the wearable sensor database 81 is defined as a model 84, and a standard index is calculated using the model 84. The calculated standard index is stored in the database A 19. The data extracted from the video sensor database 82 is defined as a model 85, and a standard index is calculated using the model 85. The calculated standard index is stored in the database A 19. The data extracted from the environment sensor database 83 is defined as a model 86, and a standard index is calculated using the model 86. The calculated standard index is stored in the database A 19.

The standard index of physical function stored in the database A 19 varies depending on data (learned data) stored in the standard database 80. For example, when the standard index calculated based on the data stored in the healthy elderly people database is stored in the database A 19, the data stored in the database A 19 can be adapted to the independence support of the elderly. Further, when the standard index calculated based on the data stored in the child education database is stored in the database A 19, the data stored in database A 19 can be adapted to a child education data set. When this data is used, improvement of child's study behavior, exercise behavior, and the like can be expected. Further, when the standard index calculated based on the data stored in the worker work behavior database is stored in the database A 19, the data stored in the database A 19 can be used to grasp a behavior of a worker and present an improvement proposal.

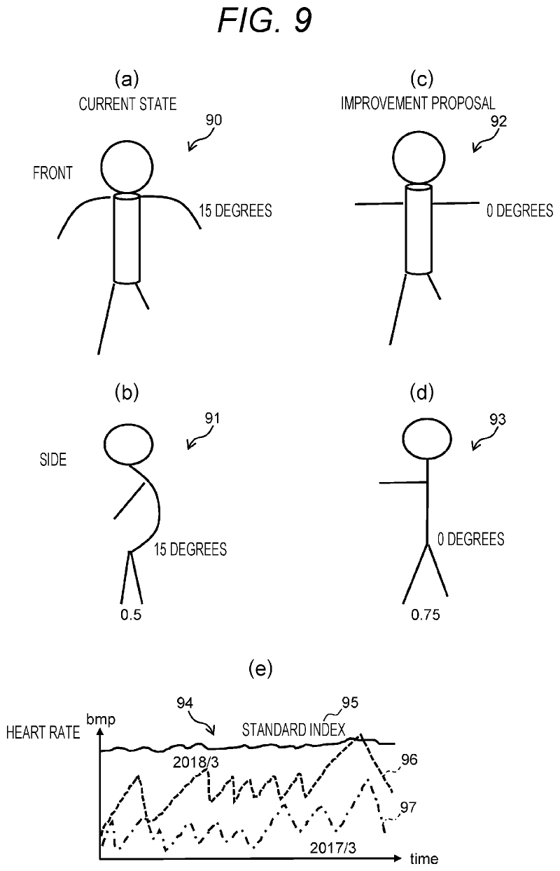

The physical function improvement proposal display unit 17 generates image information to visualize a proposal for maintenance and improvement of the physical function of the elderly person and its predictive effect based on the information proposed by the physical function improvement proposing unit 16, and displays the generated image information on the screen of the output device 2c. As the image related to the improvement proposal, what has been described in FIG. 8 can be presented in text or video. Further, it is also possible to display the predictive effect of the improvement proposal as an image in comparison with a current physical function. FIG. 9 illustrates a display example of the information proposed by the physical function improvement proposing unit 16. A display screen 90 of FIG. 9(a) is a front image illustrating the current physical function of the elderly person, and a display image 91 of FIG. 9(b) is a side image illustrating the current physical function of the elderly person. A display image 92 of FIG. 9(c) is a front image illustrating the physical function of the improvement proposal of the elderly person, and a display image 93 of FIG. 9(d) is a side image illustrating the physical function of the improvement proposal of the elderly person. From the display image 90, it can be seen that hands are not raised well with an angle of 15 degrees with respect to the horizontal plane when viewed from the front, for example, as the current state of the elderly person. From the display image 91, as the current state of the elderly person, it can be seen that the back of the elderly person is curved at 15 degrees and the step length is small when viewed from the side. As illustrated in the display images 92 and 93, it is expected that the degree of hand raising, the degree of back curve, and the step length become standard indices (0 degree, 0 degree, and 0.75), respectively, as expected effects of the improvement proposal.

Further, a display image 94 of FIG. 9(e) is an image illustrating a relationship between a standard index 95 and heart rates of the elderly person (heart rates detected by the wearable sensor 4a) 96 and 97. An improvement effect can be easily understood by displaying the heart rate (2018/3) 96 of the elderly person and the heart rate (2017/3) 97 of the elderly person with respect to the standard index 95 indicating the ideal state.

Further, the physical function improvement proposing unit 16 can grasp the physical state of the elderly person based on the data from the sensor acquisition unit 10 again in order to observe an improvement effect, and grasp the improvement effect while analyzing the physical function of the elderly person. In the case of maintaining the health of the elderly person, the physical function improvement proposing unit 16 can present to the elderly person how to prevent any change of the physical state and deterioration of the physical function and how to maintain the current state in the same flow as the processing of the improvement effect.

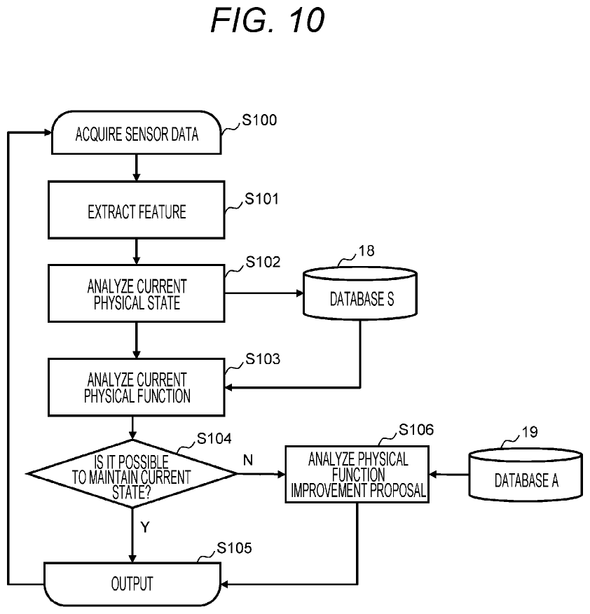

FIG. 10 illustrates a processing flow when performing health maintenance support for healthy elderly people. First, the sensor acquisition unit 10 acquires data, for example, image data and heart rate data, from the sensor (the wearable sensor 4a, the environment sensor 4b, and the video sensor 4c), and outputs the acquired data to the feature extraction unit 11 (S100). The feature extraction unit 11 extracts features (for example, foot, contour, and a low heart rate value of a person) from the data acquired from the sensor, and outputs the extracted data as feature data to the physical state analysis unit 12 (S101). The physical state analysis unit 12 uses the feature data extracted by the feature extraction unit 11 to analyze a current physical state of the elderly person, stores an analysis result in the database S 18, and outputs the analysis result to the physical function analysis unit 14 (S102).

The physical function analysis unit 14 analyzes the current physical function of the elderly person based on the analysis result of the physical state analysis unit 12 and the data stored in the database S 18 (S103), compares the analysis result of the current time with an analysis result of the previous time to determine whether the physical function of the elderly person can maintain the current state (S104). If it is determined in Step S104 that the current state can be maintained, the physical function analysis unit 14 outputs the analysis result to the physical function analysis result display unit 15 (S105), returns to the processing of Step S100, acquires data from the sensor, and repeats the next cycle. It should be noted that the image of FIG. 6 and information such as “OK as it is” are displayed on the screen of the output device 2c in Step S105 if it is determined that the current state can be maintained.

If it is determined in Step S104 that it is difficult to maintain the current state, the physical function analysis unit 14 outputs the analysis result to the physical function improvement proposing unit 16. The physical function improvement proposing unit 16 analyzes a physical function improvement proposal for maintenance or improvement of the physical function of the elderly person based on the data stored in the database A 19 and the analysis result of the physical function analysis unit 14 (S106), and outputs information indicating an analysis result (physical function improvement proposal information) to the physical function improvement proposal display unit 17 (S105). In this case, the image of FIG. 7 (the image of the physical function improvement proposal information 70) is displayed on the screen of the output device 2c in Step S105.

From the image of FIG. 6 or FIG. 7, it is possible to observe whether the elderly person maintain the health.

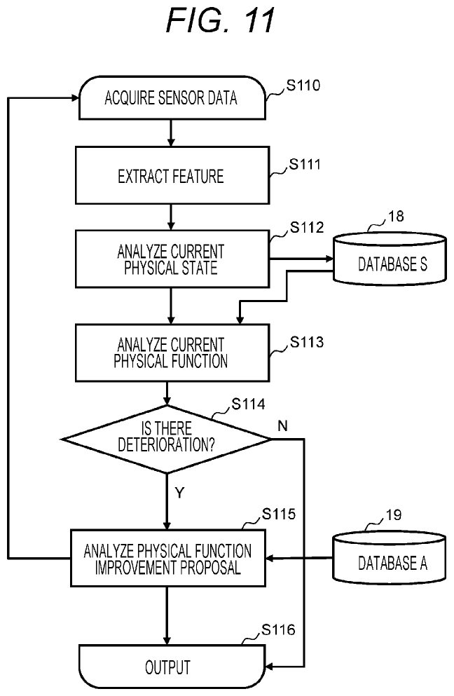

FIG. 11 illustrates a processing flow when modulation management of the elderly person is performed. First, the sensor acquisition unit 10 acquires data, for example, image data and heart rate data, from the sensor (the wearable sensor 4a, the environment sensor 4b, and the video sensor 4c), and outputs the acquired data to the feature extraction unit 11 (S110). The feature extraction unit 11 extracts features (for example, foot, contour, and a low heart rate value of a person) from the data acquired from the sensor, and outputs the extracted data as feature data to the physical state analysis unit 12 (S111). The physical state analysis unit 12 uses the feature data extracted by the feature extraction unit 11 to analyze a current physical state of the elderly person, stores an analysis result in the database S 18, and outputs the analysis result to the physical function analysis unit 14 (S112).

The physical function analysis unit 14 analyzes the current physical function of the elderly person based on the analysis result of the physical state analysis unit 12 and the data stored in the database S 18 (S113), compares the analysis result of the current time with an analysis result of the previous time to determine whether the physical function of the elderly person has deteriorated (S114). If it is determined in Step S114 that the physical function has not deteriorated, the physical function analysis unit 14 outputs the analysis result to the physical function analysis result display unit 15 (S116). The physical function analysis result display unit 15 displays, for example, the information “OK as it is” or the display image 63 of FIG. 6(b) on the screen of the output device 2c as the analysis result of the physical function analysis unit 14.

If it is determined in Step S114 that the physical function has deteriorated, the physical function analysis unit 14 outputs the analysis result to the physical function improvement proposing unit 16. The physical function improvement proposing unit 16 analyzes a physical function improvement proposal for improvement of the physical function of the elderly person based on the data stored in the database A 19 and the analysis result of the physical function analysis unit 14 (S115), returns to the processing of Step S110, acquires data from the sensor, and repeats the next cycle. Furthermore, the physical function improvement proposing unit 16 outputs information indicating the analysis result (physical function improvement proposal information) to the physical function improvement proposal display unit 17 (S116). The physical function improvement proposal display unit 17 displays, for example, the image of FIG. 7 (the image of the physical function improvement proposal information 70) on the screen of the output device 2c.

From the image of FIG. 6 or FIG. 7, it is possible to grasp the modulation of the physical function of the elderly person.

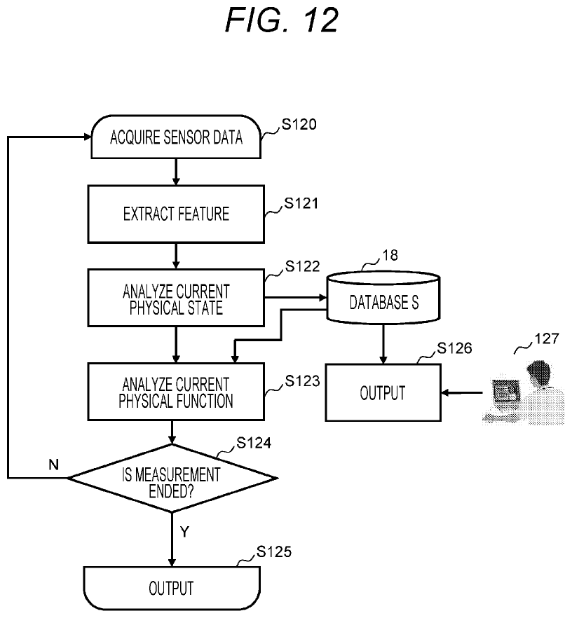

FIG. 12 illustrates a processing flow when diagnosing and treating the elderly person. First, the sensor acquisition unit 10 acquires data, for example, image data and heart rate data, from the sensor (the wearable sensor 4a, the environment sensor 4b, and the video sensor 4c), and outputs the acquired data to the feature extraction unit 11 (S120). The feature extraction unit 11 extracts features (for example, foot, contour, and a low heart rate value of a person) from the data acquired from the sensor, and outputs the extracted data as feature data to the physical state analysis unit 12 (S121). The physical state analysis unit 12 uses the feature data extracted by the feature extraction unit 11 to analyze a current physical state of the elderly person, stores an analysis result in the database S 18, and outputs the analysis result to the physical function analysis unit 14 (S122).

The physical function analysis unit 14 analyzes a current physical function of the elderly person based on the analysis result of the physical state analysis unit 12 and the data stored in the database S 18 (S123), and determines whether the measurement has ended (S124). If it is determined in Step S124 that the measurement has not ended, the physical function analysis unit 14 returns to the processing of Step S120, acquires data from the sensor, and repeats the next cycle.

If it is determined in Step S124 that the measurement has ended, the physical function analysis unit 14 ends the process of this routine (S125). It should be noted that the physical function analysis unit 14 can also output the analysis result to the physical function analysis result display unit 15 and display the analysis result on the screen of the output device 2c.

Further, when the analysis result is stored in the database S 18, the physical state analysis unit 12 outputs the data stored in the database S 18 to the physical state analysis result display unit 13 (S126). In this case, an image of the analysis result having a large granularity and a large amount of information is displayed on the screen of the output device 2c for diagnosis and treatment. Therefore, a doctor (caregiver) 127 can use the image displayed on the screen of the output device 2c for diagnosis and treatment.

It should be noted that the physical state analysis result display unit 13 and the output device 2c function as a first display unit that generates image information on the physical state analysis result or image information on diagnosis and treatment of the person based on the physical state analysis information 18b stored in the database S (first database) 18, and displays the generated image information on a display screen. Further, the physical function analysis result display unit 15 and the output device 2c function as a second display unit that generates image information on the physical function analysis result of the person based on the analysis result of the physical function analysis unit 14, and displays the generated image information on a display screen. Furthermore, the physical function improvement proposal display unit 17 and the output device 2c function as a third display unit that generates image information on the physical function improvement proposal for the person based on the physical function improvement proposal information 70 generated by the physical function improvement proposing unit 16, and displays the generated image information on a display screen.

According to the present embodiment, it is possible to provide the information for support of the independent health management. That is, the physical function improvement proposal information indicating the improvement proposal, which has been proposed to maintain or improve the physical function, is displayed as the information for support of the independent health management, and thus, the elderly person can always grasp his/her own physical function in an easy-to-understand manner. Furthermore, independence care can be performed by referring to the improvement proposal proposed to maintain or improve the physical function. As a result, the independence support can be performed so that it becomes possible to provide the independence care at home without being limited to a facility although the nursing care business has been conducted in facilities with specialized caregivers conventionally.

Further, in the conventional nursing care, it is necessary for the specialized caregiver to grasp the entire physical state of each of the elderly people, which is a heavy burden. In particular, one-on-one support requires a large amount of personnel resources, but there is a great shortage of caregivers. In regard to this, when the system according to the present embodiment is used, it is possible to support the independence of the elderly and solve the burden of nursing care and human resource problems.

Furthermore, it is possible for the healthy elderly to maintain the healthy physical function and perform independent care, and thus, the effect of improving the healthy life expectancy of the elderly can be expected.

### Second Embodiment

The present embodiment manages a plurality of elderly people by dividing them into a plurality of groups, and supports physical functions of elderly people who belong to each group, and a configuration of a physical function independence support device (server 2) is the same as that in the first embodiment. It should be noted that information on the plurality of elderly people is stored in the database S 18 in a state of being divided into the plurality of groups.

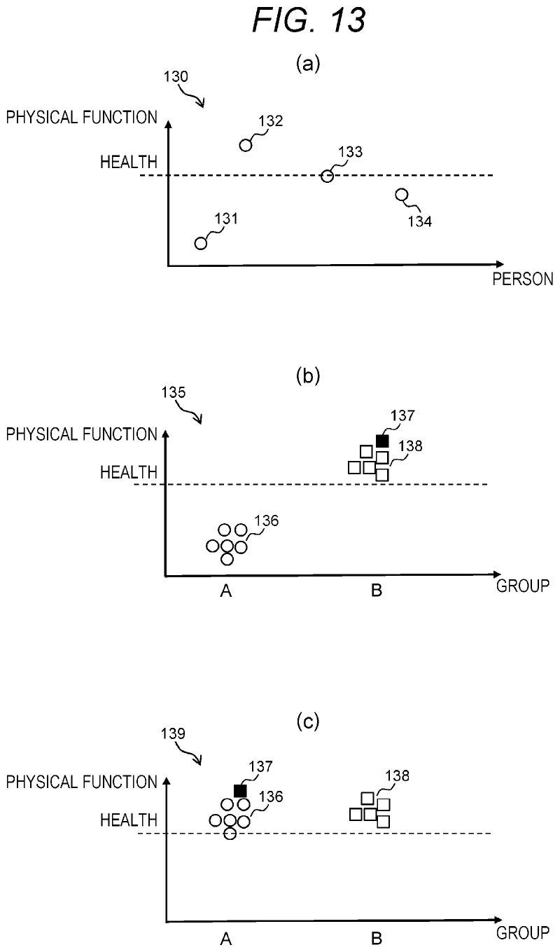

FIG. 13 illustrates a display example when performing physical function support for a group of elderly people. The elderly often lives in groups in nursing care facilities. It is possible to automatically support physical functions of those groups. First, the physical function independence support device (server 2) is used to analyze a physical function of each person, and all analysis results are plotted and output on a screen of the output device 2c. For example, as illustrated in FIG. 13(a), analysis results 131 to 134 of the respective elderly people are plotted and displayed on a display screen 130 of the physical function. Since the analysis results 131 to 134 of the respective elderly people are plotted and displayed, each of the elderly people can know the rank of the physical function in the group life and know that he/she needs to be improved more. Further, it can be pointed out to a caregiver that some of the elderly are in poor health due to slow health improvement in the group.

Further, it is possible to divide the plurality of elderly people into the plurality of groups, analyze physical functions of elderly people belonging to each group, plot the analysis results for each group, and output the analysis results on the screen of the output device 2c. A display screen 135 of FIG. 13(b) is obtained by dividing a plurality of elderly people into a group A and a group B, analyzing physical functions of elderly people belonging to each group, plotting analysis results 136 and 138 for each group, and displaying the analysis results on the screen of the output device 2c. It can be seen from the display screen 135 that the elderly people belonging to the group A as a whole have lower health (value) than the elderly people belonging to the group B. The elderly people belonging to the group B are healthy on average, and elderly people corresponding to the analysis result 137 among them are the most active and healthy. When these analysis results of the elderly people are visualized, it can be seen that, for example, the elderly people belonging to the group B are positively affected.

Meanwhile, elderly people corresponding to the analysis result 137 are moved from the group B to the group A to adjust the analysis results of the group A and the group B as illustrated in a display screen 139 of FIG. 13(c), an average value of all the elderly people belonging to the group A increases, and thus, it is possible to expect for the elderly people belonging to the group A to further improve their health. At this time, the physical functions are grasped for each group, and thus, it is possible to support the health of not only the individual but also the elderly people belonging to the group.

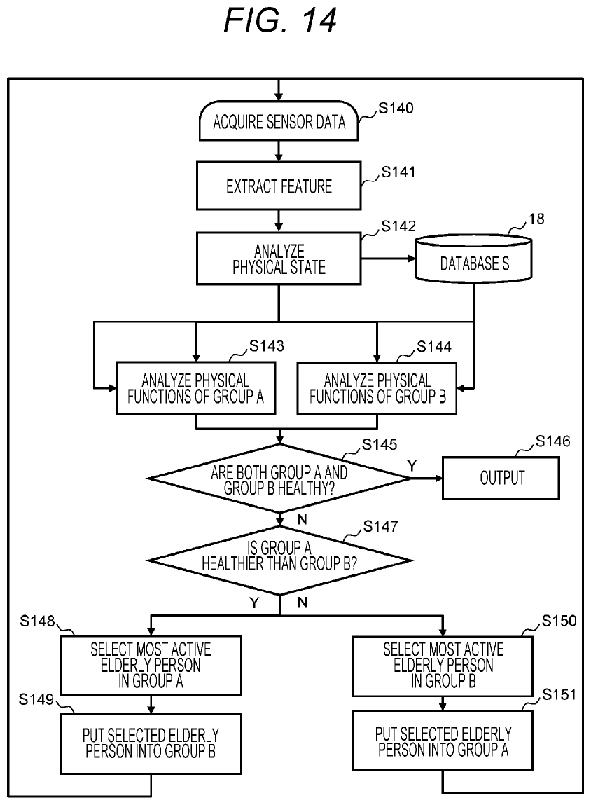

FIG. 14 illustrates a processing flow when a plurality of elderly people are divided into a plurality of groups and physical functions of the elderly people belonging to each group are analyzed. The sensor acquisition unit 10 acquires data, for example, image data and heart rate data, from the sensor (the wearable sensor 4a, the environment sensor 4b, and the video sensor 4c), and outputs the acquired data to the feature extraction unit 11 (S140). The feature extraction unit 11 extracts features (for example, foot, contour, and a low heart rate value of a person) from the data acquired from the sensor, and outputs the extracted data as feature data to the physical state analysis unit 12 (S141). The physical state analysis unit 12 uses the feature data extracted by the feature extraction unit 11 to analyze a current physical state of the elderly person, stores an analysis result in the database S 18, and outputs the analysis result to the physical function analysis unit 14 (S142).

The physical function analysis unit 14 analyzes current physical functions of the elderly people belonging to the group A based on the analysis result of the physical state analysis unit 12 and the data stored in the database S 18 (S143), and analyzes current physical functions of the elderly people belonging to the group B based on the analysis result of the physical state analysis unit 12 and the data stored in the database S 18 (S144).

Next, the physical function analysis unit 14 compares an analysis result in Step S143 and an analysis result in Step S144 with each standard index and determines whether all the elderly people belonging to the group A and the elderly people belonging to the group B are healthy (S145). If all the elderly people are determined to be healthy, the physical function analysis unit 14 outputs the analysis result in Step S143 and the analysis result in Step S144 to the output device 2c via the physical function analysis result display unit 15 (S146). For example, information such as “OK as it is” is displayed on the screen of the output device 2c. In this case, the elderly people belonging to each group can determine that the physical function does not need to be improved.

On the other hand, if it is determined in Step S145 that not all the elderly people are healthy, the physical function analysis unit 14 compares, for example, an average value of the physical functions of the elderly people belonging to the group A with an average value of the physical functions of the elderly people belonging to the group B, and determines whether the group A is healthier than the group B (S147). If it is determined in Step S147 that the group A is healthier than the group B, the physical function analysis unit 14 selects the most active elderly person (elderly person having the highest physical function) from the elderly people belonging to the group A (S148), and puts the selected elderly person into the group B (S149). Thereafter, the physical function analysis unit 14 returns to the processing of Step S140 and repeats the next cycle. At this time, the physical function analysis unit 14 evaluates physical functions of elderly people in each group again using the group A in which the elderly person having the highest physical function has been decreased and the group B in which the elderly person having the highest physical function has been increased as new groups A and B.

If it is determined in Step S147 that the group A is not healthier than the group B, the physical function analysis unit 14 selects the most active elderly person (elderly person having the highest physical function) from the elderly people belonging to the group B (S150), and puts the selected elderly person into the group A (S151). Thereafter, the physical function analysis unit 14 returns to the processing of Step S140 and repeats the next cycle. At this time, the physical function analysis unit 14 evaluates physical functions of elderly people in each group again using the group A in which the elderly person having the highest physical function has been increased and the group B in which the elderly person having the highest physical function has been decreased as new groups A and B.

According to the present embodiment, it is possible to expect the effect of improving the overall health of groups including active and healthy elderly people by putting active and healthy elderly people in another group. Further, the elderly people can help each other within the group and between the groups so that improvement of an independence effect of each elderly person can be expected. It should be noted that group information of each elderly person can be designated in advance when grouping elderly people belonging to each group. Further, an active and healthy elderly person can be selected on the screen when the screen of FIG. 13(b) is displayed in the case of selecting the active and healthy elderly person.

### Third Embodiment

The present embodiment supports physical functions of a plurality of children, and a configuration of a physical function independence support device (server 2) is the same as that in the first embodiment. It should be noted that information on the plurality of children is stored in the database S 18 and the database A 19.

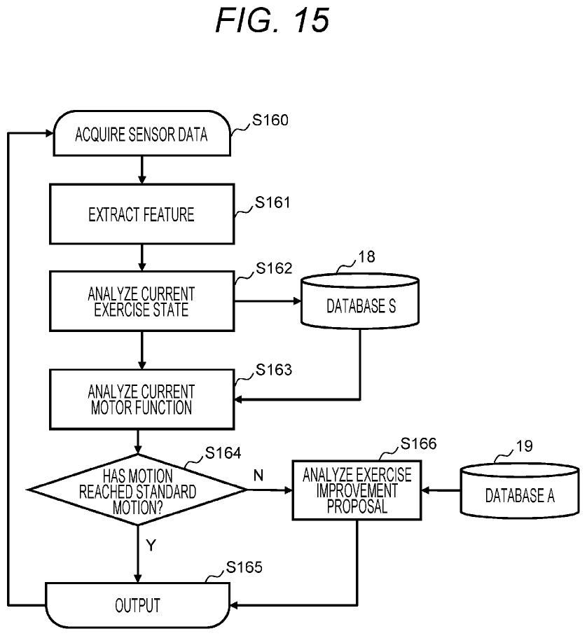

FIG. 15 illustrates a processing flow when performing motor function support for exercise training of a child. First, the sensor acquisition unit 10 acquires data, for example, image data and acceleration data, from the sensor (the wearable sensor 4a, the environment sensor 4b, and the video sensor 4c), and outputs the acquired data to the feature extraction unit 11 (S160). The feature extraction unit 11 extracts features (for example, a skeleton and a speed of a child) from the data acquired from the sensor, and outputs the extracted data as feature data to the physical state analysis unit 12 (S161). The physical state analysis unit 12 uses the feature data extracted by the feature extraction unit 11 to analyze a current exercise state of the child, stores an analysis result in the database S 18, and outputs the analysis result to the physical function analysis unit 14 (S162).

The physical function analysis unit 14 analyzes a current motor function of the child based on the analysis result of the physical state analysis unit 12 and the data stored in the database S 18 (S163), and compares the current data with data indicating a standard motion to determine whether a motion of the child has reached the standard motion (S164). If it is determined in Step S164 that the standard motion has been reached, the physical function analysis unit 14 outputs the analysis result to the physical function analysis result display unit 15 (S165), returns to the processing of Step S160, acquires data from the sensor, and repeats the next cycle. It should be noted that information such as “OK as it is” is displayed on the screen of the output device 2c if the physical function analysis unit 14 determines that the standard motion has been reached.

If it is determined in Step S164 that the standard motion has not been reached, the physical function analysis unit 14 outputs the analysis result to the physical function improvement proposing unit 16. The physical function improvement proposing unit 16 analyzes an exercise improvement proposal (motor function improvement proposal) for maintenance or improvement of the motor function of the child based on the data stored in the database A 19 and the analysis result of the physical function analysis unit 14. (S166), and outputs information indicating an analysis result (motor function improvement proposal information) to the physical function improvement proposal display unit 17 (S165). In this case, an image generated based on the motor function improvement proposal information is displayed on the screen of the output device 2c. It should be noted that the physical function improvement proposing unit 16 generates information to support exercise training of a person as the information belonging to the physical function improvement proposal information when information on the exercise training of the person is included in the analysis result of the physical function analysis unit 14 and standard index information indicating a standard index for the exercise training of the person is stored in the database A 19.

According to the present embodiment, by observing the image displayed on the screen of the output device 2c, even the child can compare his/her own motion with the standard motion, and as a result, can grasp a point that needs to be improved. Further, this system can systematically support the motor function for the exercise training of the children, and thus, the burden on the school side such as a teacher can be reduced. Further, this system analyzes an exercise improvement proposal and displays the analysis result when the current motor function of the child has not reached the standard motion, and thus, the child can repeat the motion until reaching the standard motion.

Further, when this system is applied to support the independence of the physical function of the child, it is possible to expect the improvement of the motor function and the health function of the child in accordance with the growth of the child. Further, the reduction in the burden of the school can be expected by supporting the independence of the child systematically.

### Fourth Embodiment

The present embodiment supports physical functions of a plurality of workers, and a configuration of a physical function independence support device (server 2) is the same as that in the first embodiment. It should be noted that information on the plurality of workers is stored in the database S 18 and the database A 19.

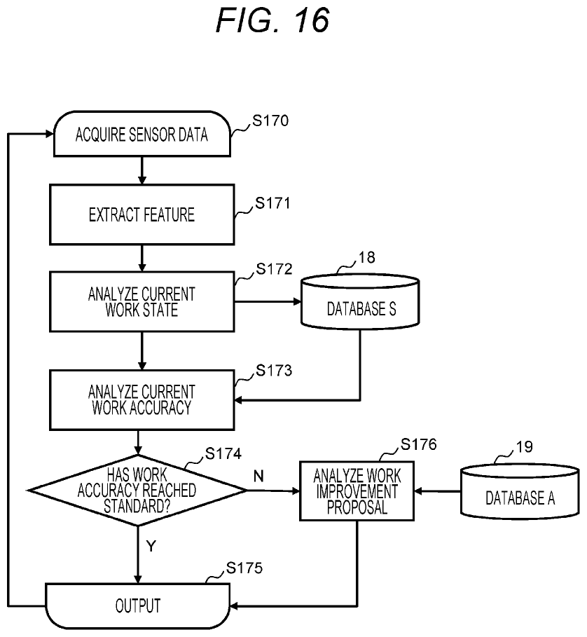

FIG. 16 illustrates a processing flow when performing physical function support for work of a worker. First, the sensor acquisition unit 10 acquires data, for example, image data and heart rate data, from the sensor (the wearable sensor 4a, the environment sensor 4b, and the video sensor 4c), and outputs the acquired data to the feature extraction unit 11 (S170). The feature extraction unit 11 extracts features (for example, a hand position, an angle, a line of sight, and the like of a person) from the data acquired from the sensor, and outputs the extracted data as feature data to the physical state analysis unit 12 (S171). The physical state analysis unit 12 uses the feature data extracted by the feature extraction unit 11 to analyze a current work state of the worker, stores an analysis result in the database S 18, and outputs the analysis result to the physical function analysis unit 14 (S172).

The physical function analysis unit 14 analyzes current work accuracy of the worker based on the analysis result of the physical state analysis unit 12 and the data stored in the database S 18 (S173), and compares the current analysis result with standard work accuracy to determine whether the work accuracy of the worker has reached the standard (standard work accuracy) (S174). If it is determined in Step S174 that the standard has been reached, the physical function analysis unit 14 outputs the analysis result to the physical function analysis result display unit 15 (S175), returns to the processing of Step S170, acquires data from the sensor, and repeats the next cycle. It should be noted that information such as “OK as it is” is displayed on the screen of the output device 2c if it is determined that the standard has been reached.

If it is determined in Step S174 that the standard has not been reached, the physical function analysis unit 14 outputs the analysis result to the physical function improvement proposing unit 16. The physical function improvement proposing unit 16 analyzes a work improvement proposal (work accuracy improvement proposal) for maintenance or improvement of the work accuracy of the worker based on the data stored in the database A 19 and the analysis result of the physical function analysis unit 14 (S176), and outputs information indicating an analysis result (work accuracy improvement proposal information) to the physical function improvement proposal display unit 17 (S175). In this case, an image generated based on the work accuracy improvement proposal information is displayed on the screen of the output device 2c. It should be noted that the physical function improvement proposing unit 16 generates information to support work training of a person so as to belong to the physical function improvement proposal information when information on the work training of the person is included in the analysis result of the physical function analysis unit 14 and standard index information indicating a standard index for the work training of the person is stored in the database A 19.

This system analyzes the work improvement proposal and displays the analysis result when the current work accuracy of the worker does not reach the standard (standard work accuracy), and thus, the worker can repeat a motion until the work accuracy reaches the standard.

According to the present embodiment, the system of the present embodiment can be applied to the training of workers, and to present the improvement proposal using data of one expert as a standard can be used for training of several workers. Therefore, training cost can be reduced. Further, a work state to be analyzed can be also adjusted depending on a work item, and the training effect can be improved. Furthermore, whether the standard has been reached is systemically analyzed, it is possible to improve the quality of the work, and improvement of the quality of a product can be also expected.

Similarly, when the system of the present embodiment is applied to support independence of a physical function of a worker, improvement of a work behavior of the worker and improvement of work efficiency can be expected. Further, it is possible to systematically support training of an operator by digitizing the field knowledge and applying the digitized information to the system.

It should be noted that the present invention is not limited to the above-described embodiments, but includes various modifications. For example, the image information displayed on the output device 2c can be transmitted to the user terminal 4 via the network 3 and displayed on the display of the user terminal 4. In this case, the user such as the elderly person can check a health condition by looking at the image displayed on the display of the user terminal 4. The above-described embodiments have been described in detail in order to explain the present invention in an easy-to-understand manner, and are not necessarily limited to those having all the configurations described. Further, a part of the configuration of a certain embodiment can be replaced with the configuration of another embodiment, and the configuration of another embodiment can be added to the configuration of a certain embodiment. Further, it is possible to add/delete/replace other configurations with respect to a part of the configurations of the respective embodiments.

Further, each of the above-described configurations, functions, and the like may be partially or entirely realized by hardware, for example, by designing an integrated circuit. Further, the above-described respective configurations, functions and the like may be realized by software by the processor interpreting and executing a program for realizing the respective functions. Information such as programs, tables, and files that realize each function is stored in a memory, hard disk, recording device such as SSD (Solid State Drive), or IC (Integrated Circuit) card, SD (Secure Digital) memory card, DVD (It can be recorded and placed on a recording medium such as a Digital Versatile Disc).

### REFERENCE SIGNS LIST

1 physical function independence support system
2 server
2a CPU
2b input device
2c output device
2d communication device
2e storage device
3 network
4 user terminal
4a wearable sensor
4b environment sensor
4c video sensor
10 sensor acquisition unit
11 feature extraction unit
12 physical state analysis unit
13 physical state analysis result display unit
14 physical function analysis unit
15 physical function analysis result display unit
16 physical function improvement proposing unit
17 physical function improvement proposal display unit
18 database S
19 database A
50 physical function improvement analysis unit
51 motor function analysis unit
52 daily activity analysis unit
80 standard database
81 wearable sensor database
82 video sensor database
83 environment sensor database
84 to 86 model

1. A physical function independence support device, which transmits and receives information to and from one or more sensors that detect at least people, comprising:
an acquisition unit that acquires physical state information indicating physical states of the people from the sensor;
a physical function analysis unit that analyzes a change in physical functions of the people based on a time-series change of the physical state information acquired by the acquisition unit; and
a physical function improvement proposing unit that generates and outputs physical function improvement proposal information indicating an improvement proposal of the physical function with respect to the change in the physical functions of the people based on an analysis result of the physical function analysis unit.

2. The physical function independence support device according to claim 1, further comprising a feature extraction unit that extracts feature information, which is a feature, from the physical state information acquired by the acquisition unit,
wherein the physical function analysis unit analyzes the change in the physical functions of the people based on a time-series change of the feature information extracted by the feature extraction unit.

3. The physical function independence support device according to claim 2, further comprising a first database that stores the physical state information acquired by the acquisition unit in a time-series manner,
wherein the physical function analysis unit analyzes the change in the physical functions of the people based on the feature information extracted by the feature extraction unit and the physical state information stored in the first database.

4. The physical function independence support device according to claim 1, further comprising:
a feature extraction unit that extracts feature information, which is a feature, from the physical state information acquired by the acquisition unit;
a physical state analysis unit that analyzes physical states of the people based on the feature information extracted by the feature extraction unit; and
a first database that stores an analysis result of the physical state analysis unit in a time-series manner as physical state analysis information,
wherein the physical function analysis unit analyzes the change in the physical functions of the people based on the analysis result of the physical state analysis unit and the physical state analysis information stored in the first database.

5. The physical function independence support device according to claim 4, wherein in the first database, information indicating a posture, a behavior, and physical burden/fatigue is stored in time-series manner as the physical state analysis information.

6. The physical function independence support device according to claim 4, wherein
the physical function analysis unit includes:
a physical function improvement analysis unit that analyzes whether the physical functions of the people have been improved based on the analysis result of the physical state analysis unit and the physical state analysis information stored in the first database;
a motor function analysis unit that analyzes a change in motor functions of the people based on the analysis result of the physical state analysis unit and the physical state analysis information stored in the first database; and
a daily activity analysis unit that analyzes a change in daily activities of the people based on the analysis result of the physical state analysis unit and the physical state analysis information stored in the first database.

7. The physical function independence support device according to claim 5, further comprising a second database that stores standard index information indicating a standard index of the physical state analysis information,
wherein the physical function improvement proposing unit compares the analysis result of the physical function analysis unit with the standard index information, and generates the physical function improvement proposal information based on a comparison result.

8. The physical function independence support device according to claim 7, wherein the physical function improvement proposing unit generates information to support health maintenance of the people as information belonging to the physical function improvement proposal information when information on the health maintenance of the people is included in the analysis result of the physical function analysis unit and the standard index information is stored in the second database.

9. The physical function independence support device according to claim 7, wherein the physical function improvement proposing unit generates information to support exercise training of the people as information belonging to the physical function improvement proposal information when information on the exercise training of the people is included in the analysis result of the physical function analysis unit and the standard index information is stored in the second database.

10. The physical function independence support device according to claim 7, wherein the physical function improvement proposing unit generates information to support work training of the people so as to belong to the physical function improvement proposal information when information on the work training of the people is included in the analysis result of the physical function analysis unit and the standard index information is stored in the second database.

11. The physical function independence support device according to claim 4, further comprising a first display unit that generates image information on diagnosis and treatment of the people based on the physical state analysis information stored in the first database, and displays the generated image information on a display screen.

12. The physical function independence support device according to claim 4, further comprising a second display unit that generates image information on physical state analysis results of the people based on the analysis result of the physical state analysis unit, and displays the generated image information on a display screen.

13. The physical function independence support device according to claim 8, further comprising a third display unit that generates image information on physical function improvement proposals for the people is generated based on the physical function improvement proposal information generated by the physical function improvement proposing unit, and displays the generated image information on a display screen.

14. The physical function independence support device according to claim 4, wherein the physical function analysis unit divides the people into a plurality of groups based on the analysis result of the physical state analysis unit and the physical state analysis information stored in the first database, and analyzes a change in physical functions of people who belong to each of the groups.

15. A physical function independence support method for transmitting and receiving information to and from one or more sensors that detect at least people, the method comprising:
an acquisition step of acquiring physical state information indicating physical states of the people from the sensor;
a physical function analysis step of analyzing a change in physical functions of the people based on a time-series change of the physical state information acquired in the acquisition step; and
a physical function improvement proposal step of generating and outputting physical function improvement proposal information indicating an improvement proposal of the physical function with respect to the change in the physical functions of the people based on an analysis result in the physical function analysis step.

16. The physical function independence support device according to claim 9, further comprising a third display unit that generates image information on physical function improvement proposals for the people is generated based on the physical function improvement proposal information generated by the physical function improvement proposing unit, and displays the generated image information on a display screen.

17. The physical function independence support device according to claim 10, further comprising a third display unit that generates image information on physical function improvement proposals for the people is generated based on the physical function improvement proposal information generated by the physical function improvement proposing unit, and displays the generated image information on a display screen.
